(19) United States
(12) Patent Application Publication
(10) Pub. No.: US 2004/0002101 A1
(43) Pub. Date: Jan. 1, 2004

(54) Method and kit for discovering nucleic acids that encode desired functions

(75) Inventors: Daniel Dupret, Calvisson (FR); Jean-Michel Masson, Toulouse (FR); Fabrice Lefevre, Nimes (FR)

Correspondence Address: HUNTON & WILLIAMS, INTELLECTUAL PROPERTY DEPARTMENT, 1900 K STREET, N.W., SUITE 1200, WASHINGTON, DC, 20006-1109, US

(73) Assignee: Proteus S.A.

(21) Appl. No.: 10/411,275

(22) Filed: Apr. 11, 2003

Related U.S. Application Data
(63) Continuation-in-part of application No. 10/288,591, filed on Nov. 6, 2002, now abandoned.
Application No. 10/288,591 is a continuation-in-part of application No. 09/722,392, filed on Nov. 28, 2000, now Pat. No. 6,514,703.
Application No. 09/722,392 is a continuation of application No. PCT/FR99/01972, filed on Aug. 11, 1999.

(30) Foreign Application Priority Data
Aug. 12, 1998 (FR) 98/10337

(51) Int. Cl. C12Q 1/68

(52) U.S. Cl. 435/6

(57) ABSTRACT

Method and kit for using in vitro expression to discover nucleic acids that encode desired functions. Either the existence, presence, identity, properties or function of one or more of the nucleic acids from the sample is unknown to at least the experimenter performing the method or using the kit.

### CROSS-REFERENCE TO RELATED APPLICATIONS

This application is a CIP of U.S. application Ser. No. 10/288,591, filed Nov. 6, 2002, which is a CIP of U.S. application Ser. No. 09/722,392, filed Nov. 28, 2000, now U.S. Pat. No. 6,514,703B1. This application also claims the benefit of PCT Application No. PCT/FR99/01972, filed Aug. 11, 1999; and French Patent Application No. FR98/10337, filed Aug. 12, 1998. All of the foregoing applications and patent are herein incorporated by reference in their entireties.

### BACKGROUND OF THE INVENTION

In their search for new genes, proteins, and protein activities, molecular biologists choose from among several approaches for isolating and screening genes. One approach, “genomics,” conventionally entails sequencing a part or all of an organism's genome. The homology of each generated sequence is then compared to that of known reference sequences from a data bank of potential coding sequences. When the homology is sufficiently high, the generated sequences are subcloned and expressed to verify that they encode the desired protein. Thus, conventional genomics is limited to identifying sequences that are closely homologous to reference sequences.

Another approach, “proteomics,” conventionally entails: extracting proteins expressed by a microorganism, purifying those proteins, and detecting which purified protein fraction exhibits the desired function Proteomics is limited to detecting proteins induced in the starting microorganism. Proteomics also provides no link between the purified protein that exhibits the desired function and the nucleic acid that encodes it. To identify the nucleic acid that encodes the desired function, researchers who use proteomics must resort to one of two techniques. One technique is to microsequence the purified protein to construct degenerate primers to amplify and isolate the putative nucleic acid. The nucleic acid is then verified as the one that encodes the desired protein function. Another technique entails using antibodies specific for the purified protein to screen an expression library for a nucleic acid associated with the desired function. Both techniques are intricate and slow.

A third approach for finding new genes and proteins, “expression cloning,” conventionally entails: extracting DNA from a starting microorganism, fragmenting the DNA, inserting it into an expression vector, and transforming the vector in a host selected for its ability to express the genes of the starting microorganism. Expression cloning is useful when the host's genome is compatible with the vector's (e.g., similar codon usage, similar GC percentage). When starting with many microorganisms of varied genetic origin, however, its usefulness decreases because the hosts will not express heterologous genes.

The prior art's reliance on in vivo procedures for isolating and screening genes entails a variety of additional disadvantages:

Cellular toxicity of transcription and translation products, which can induce genetic recombinations;

Non-representative samples in the library;

High consumption of time;

High variation of expression levels, e.g., due to a cell's physiological state when the procedure employs the original cellular extracts;

Codon usage problems;

Refolding and post-translation problems; and

Difficulty of automation.

Although in vitro expression has been known since the early 1960s, the prior art has not developed in vitro expression in ways that befit screening for new genes and proteins. Thus, in Jermutus et al, Current Opinion in Biotechnology 1998, 9:534-548, the authors comment on in vitro expression as follows: “The impact of this technology is still limited by comparatively low yields and is currently used only for analytical purposes.” Jermutus et al, p.541, 1st col., 2nd para., lines 7-9. The authors also discuss problems with the reliability and specificity of in vitro expression, which derive in part from protein and mRNA degradation and depletion of reaction energy sources. These problems, which exist despite the development of continuous-flow cell-free expression systems, have pointed researchers away from using in vitro expression for screening.

### SUMMARY OF THE INVENTION

The present invention uses in vitro expression to identify unknown nucleic acids that are potentially present in biological, genomic or cDNA samples and that potentially encode or are associated with desired functions. As used herein, in vitro means outside a living organism or cell. Cellular hosts, if they are used at all, are used only for the isolation and amplification of the nucleic acids. Consequently, the invention avoids the problems of in vivo expression. The invention's independence from in vivo expression enables researchers to find desired proteins without having to resolve problems related to culture and cell physiology. Thus, the invention is particularly useful for identifying nucleic acids that express cytotoxic proteins. The invention can also assure a link between the protein found to exhibit the desired function and the corresponding nucleic acid that encodes the protein.

In one embodiment, the invention is a method of discovering nucleic acids, in a biological, genomic or cDNA sample, that are associated with a pre-selected desired function. This embodiment includes the following steps:

preparing nucleic acids from the sample, wherein at least one of the prepared nucleic acids is unknown to the experimenter performing the method;

separating the prepared nucleic acids;

treating the separated nucleic acids in vitro to obtain transcripts;

optionally treating the transcripts in vitro to obtain proteins;

testing the transcripts or proteins for association with the desired function; and

identifying the nucleic acid that encodes the transcript or protein that is associated with the desired function.

In another embodiment, the invention is a method of discovering nucleic acids, in a biological or genomic sample, that encode a desired protein function. This embodiment includes the following steps:

selecting a specific desired function;

preparing nucleic acids from the sample, wherein the existence, presence, identity, properties, or function of at least one of the prepared nucleic acids is unknown to the experimenter performing the method;

separating the prepared nucleic acids;

treating the separated nucleic acids in vitro to obtain transcripts;

treating the transcripts in vitro to obtain proteins;

testing the proteins for the desired function; and

identifying the nucleic acid that encodes the protein that exhibits the desired function.

Note that, as used in the embodiment above and throughout this application, the phrase “function of at least one of the prepared nucleic acids” and equivalent phrases are shorthand for the function exhibited by or associated with the transcript or protein encoded by the prepared nucleic acids. In other words, the phrase does not literally refer to the function of a prepared nucleic acid itself; rather, the phrase refers to the function of its expression product. Also, the “presence” of the prepared nucleic acids refers to their presence in the sample.

In another embodiment, the invention is a method of discovering nucleic acids, in a biological or genomic sample, that encode a desired RNA function. This embodiment includes the following steps:

selecting a specific desired RNA function;

preparing nucleic acids from the sample, wherein the existence, presence, identity, properties or function of at least one of the prepared nucleic acids is unknown to the experimenter performing the method;

separating the prepared nucleic acids;

treating the separated nucleic acids in vitro to obtain transcripts;

testing the transcripts for the desired function; and

identifying the nucleic acid that encodes the transcript that exhibits the desired function.

In a more preferred version of the above embodiment, the desired function is a desired tRNA, Tm RNA, si RNA or ribozyme function. Still more preferably, the desired function is a desired tRNA or ribozyme function.

More preferably, the existence, presence, identity, properties and/or function of more than one of the prepared nucleic acids is unknown to the experimenter performing the method or unknown to science. Even more preferably, at least a majority of the prepared nucleic acids are unknown to the experimenter or science.

Additional preferred embodiments include one or more of the following features:

the sample is a biological sample that includes or is extracted from at least one species or strain of organism; the organism is unknown to the experimenter; the existence, presence, properties and/or identity of the organism is unknown to the experimenter or science;

the sample includes or derives from one or more extremophiles; the extremophiles are acidophiles that can grow in a pH lower than 2; the extremophiles are alkalinophiles that can grow in a pH higher than 11; the extremophiles are psychrophiles that can grow in temperatures below 0° C. to 4° C.; the extremophiles are thermophiles that can grow in temperatures above 60° C. to 70° C.; the extremophiles are barophiles that can grow at the pressures at the bottom of the ocean; the extremophiles are halophiles that can grow in a salt concentration of approximately 25-32 percent; the extremophiles are radiophiles that can grow in areas saturated with nuclear waste; the extremophiles are oligotrophs; the extremophiles are anaerobes; the extremophiles fall into more than one category of extremophile; the extremophiles are thermophilic barophiles that can grow in undersea thermal vents;

separating the prepared nucleic acids initially includes inserting the prepared nucleic acids into plasmidic vector molecules to form recombinant vectors, wherein the plasmidic vector molecules include a cloning site and an RNA polymerase promoter on at least one side of the cloning site; separating the prepared nucleic acids further includes separating the recombinant vectors with a microorganism in which said promoter does not function;

the step of treating the separated nucleic acids in vitro to obtain proteins is performed with a translation extract derived from an organism or organisms from the same family as the organism or organisms from which the sample derives; the translation extract is derived from an organism or organisms from the same genera as the organism or organisms from which the sample derives; the translation extract is derived from an organism or organisms from the same species as the organism or organisms from which the sample derives;

the translation extract is prepared from eukaryotic cells;

one or more of the prepared nucleic acids includes an amber codon and the translation extract includes a tRNA suppressor specific for that codon;

the step of treating the separated nucleic acids in vitro to obtain proteins is performed with a universal translation extract derived from an organism or organisms from a different family, genera or species than the organism or organisms from which the sample derives;

the step of treating the separated nucleic acids in vitro to obtain transcripts and the step of treating the transcripts in vitro to obtain proteins are coupled and occur simultaneously in the same reaction mixture;

the step of treating the separated nucleic acids in vitro to obtain transcripts and the step of treating the transcripts in vitro to obtain proteins are temporally or physically distinct.

the desired function is an enzymatic activity; the enzymatic activity is a member selected from the group consisting of oxidoreductase activity, transferase activity, hydrolase activity, lyase activity, isomerase activity and ligase activity; the desired RNA function is a member selected from the group consisting of a ribozyme function, a tRNA function, a Tm RNA function and a SI RNA function; the desired RNA function is a ribozyme function and the ribozyme function is an endonuclease function;

the protein associated with or exhibiting the desired function is physically free from the nucleic acid that encodes it during and after the step of testing the protein for the desired function;

the desired function is a function other than a binding affinity;

An alternative embodiment of the invention includes an apparatus, device or kit for carrying out most or all steps of the method of the invention. Preferably, the apparatus, device or kit allows one or more of the steps to be automated. For example, in one embodiment the invention is a kit for discovering nucleic acids, in a biological, genomic or cDNA sample, associated with pre-selected functions. This embodiment includes the following features:

one or more containers;

reagents for preparing nucleic acids from the sample, wherein at least one of the prepared nucleic acids is unknown to the experimenter using the kit;

vectors in which the prepared nucleic acids can be inserted;

reagents for inserting the prepared nucleic acids into the vectors to form recombinant vectors;

reagents for separating the recombinant vectors;

reagents for transcribing the separated recombinant vectors in vitro to obtain transcripts;

optionally, reagents for translating the transcripts in vitro to obtain proteins; and

reagents for testing the transcripts or proteins for a pre-selected function.

In another embodiment, the invention is a kit for discovering nucleic acids, in a biological or genomic sample, that encode desired functions. This embodiment includes the following features:

one or more containers;

reagents for preparing nucleic acids from the sample, wherein the existence, presence, identity, properties or function of at least one of the prepared nucleic acids is unknown to the experimenter using the kit;

vectors in which the prepared nucleic acids can be inserted;

reagents for inserting the prepared nucleic acids into the vectors to form recombinant vectors;

reagents for separating the recombinant vectors;

reagents for transcribing the separated recombinant vectors in vitro to obtain transcripts;

reagents for translating the transcripts in vitro to obtain proteins; and

reagents for testing the proteins for a desired function.

In another embodiment, the invention is a kit for discovering nucleic acids, in a biological or genomic sample, that encode desired RNA functions. This embodiment includes the following features:

one or more containers;

reagents for preparing nucleic acids from the sample, wherein the existence, presence, identity, properties or function of at least one of the prepared nucleic acids is unknown to the experimenter using the kit;

vectors in which the prepared nucleic acids can be inserted;

reagents for inserting the prepared nucleic acids into the vectors to form recombinant vectors;

reagents for separating the recombinant vectors;

reagents for transcribing the separated recombinant vectors in vitro to obtain transcripts; and

reagents for testing the transcripts for a desired RNA function.

In another embodiment, the invention is a kit for discovering nucleic acids, in a biological or genomic sample, that encode desired functions. This embodiment includes the following features:

one or more containers;

nucleic acids prepared from the sample, wherein the existence, presence, identity, properties or function of more than one of the prepared nucleic acids is unknown to the experimenter using the kit;

recombinant vectors in which the unknown nucleic acids have been inserted;

transcripts of the recombinant vectors; and

one of either (i) reagents for translating the transcripts, (ii) proteins translated from the transcripts, (iii) reagents for testing the proteins for a desired function, (iv) or (v) a combination of any of (i)-(iv).

In still another embodiment, the invention is a kit for discovering nucleic acids, in a biological or genomic sample, that encode desired RNA functions. This embodiment includes the following features:

one or more containers;

nucleic acids prepared from the biological or genomic sample, wherein the existence, presence, identity, properties or function of ore than one of the prepared nucleic acids is unknown to the experimenter using the kit;

recombinant vectors in which the unknown nucleic acids have been inserted;

transcripts of the recombinant vectors; and

reagents for testing the transcripts for a desired RNA function.

Other advantages and characteristics of the invention will appear from the examples of carrying out the invention that follow and that refer to the attached drawings.

### BRIEF DESCRIPTION OF DRAWINGS

FIG. 1 is a schematic representation of an example of an embodiment of the invention.

FIG. 2 represents an example of a cloning vector for the construction of a library for use with the invention.

FIG. 3 represents an example of the plasmids pADH, pTEM, pET26-Tfu2, and pLIPet pGFP, which can be used with various embodiments of the invention.

FIG. 4 represents the activity of the intein 2 of a DNA polymerase of Thermococcus fumicolans (Tfu) obtained in vitro by expression cloning. Track A: molecular weigh marker 1 Kb. Track B: reaction without enzyme. Track C: reaction with intein 2.

FIG. 5 represents the activity of the GFP produced by in vitro protein expression reaction (Emission of fluorescence followed by an exposure at about 400 nm) with mesophilic translation extracts. Tube A: in vitro protein expression reaction with water (control). Tube B: in vitro protein expression reaction with the vector pGFP.

FIG. 6 represents the activity of the intein 2 of the Tfu DNA polymerase produced by in vitro protein expression reaction with mesophilic-type translation extracts. Track 1: molecular weigh marker 1 Kb. Track 2: T-(reaction without enzyme). Track 3: Blank (reaction with in vitro protein expression extract without DNA). Track 4: T⁺ (reaction with intein 2 produced in vitro). Track 5: Test (reaction with intein 2 produced by in vitro protein expression reaction (pET26-Tfu2)).

FIG. 7 represents the detection of the activity of a thermophilic enzyme by using a mesophilic translation extract containing said activity. Tube A: in vitro protein expression reaction with water (control) incubated at 37° C., Tube B: in vitro protein expression reaction incubated at 37° C., Tube C: in vitro protein expression reaction with water (control) incubated at 70° C. after centrifugation. Tube D: in vitro protein expression reaction incubated at 70° C. after centrifugation.

FIG. 8 shows the detection of an esterase function using a Pyrococcus horikoshii cell free extract.

FIG. 9 shows detection of an esterase function using an E. coli cell free extract.

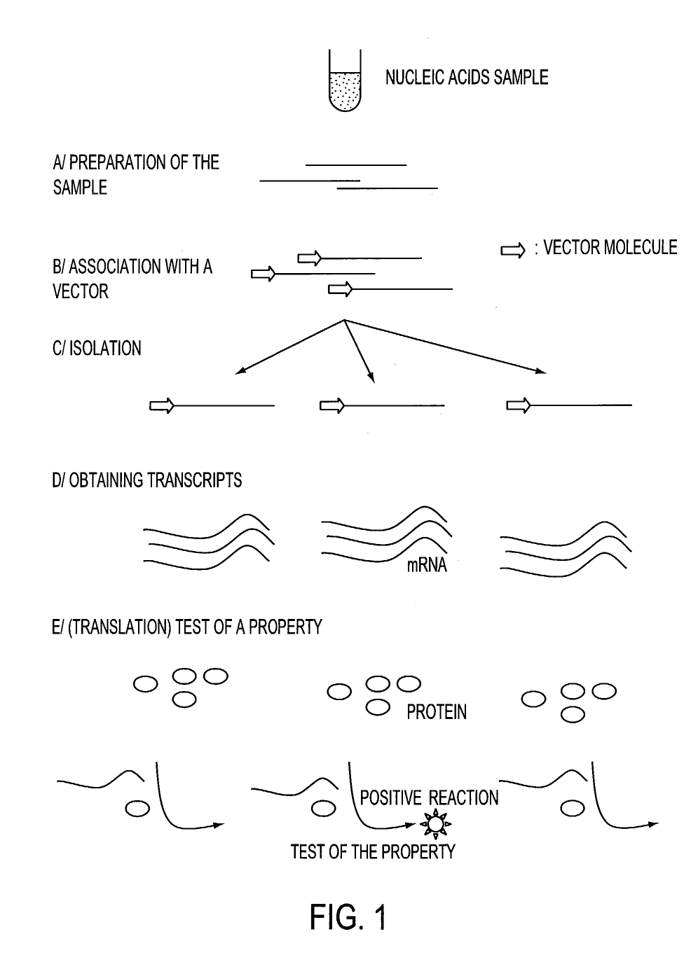

### DETAILED DESCRIPTION OF PREFERRED EMBODIMENTS

1. Advantages of Present Invention

In genomics, all or part of an organism's genome is sequenced and the sequences are compared to known sequences in a database. Based on the comparison, putative functions are assigned to the sequences. To verify the putative functions, the sequences are subcloned and expressed to ensure that they encode proteins that exhibit the putative function. Unlike genomics, the invention can identify nucleic acids in a genomic or cDNA library that are unknown to the experimenter without first sequencing everything in the library. When applied to the entire genome of an organism, the invention can characterize the entire phenotype of the organism, a concept the inventors refer to as “Phenomics.”

In proteomics and expression cloning, the nucleic acid and host must share a homologous origin. If their origins are heterologous, the host will be unable to express the protein. The invention lacks these drawbacks. As such, the invention is particularly suited to identifying nucleic acids unknown to the experimenter in biological samples that contain numerous diverse nucleic acids. The invention is especially suited to identifying nucleic acids unknown to the experimenter in biological samples that contain multiple organisms, because the invention avoids the isolation of each microorganism in the sample. Isolating each microorganism in a sample recovers only a few percent (e.g., 5 percent) of the genetic diversity in the sample, and prior art methods spend several weeks or even months screening the strains to find an interesting protein. Certain embodiments of the invention, on the other hand, can screen nearly 100 percent of the genetic diversity in as little as 5 to 10 days or less, in part because the invention is more amenable to automation than the prior art.

Like the present invention, some prior art techniques, such as phage display and ribosome or polysome display, provide a link between the desired activity and the sequence that encodes it. Phage display, however, relies on the creation of in vivo nucleic acid libraries and is therefore subject to the disadvantages of in vivo expression discussed earlier. In the prior art, ribosome or polysome display (hereafter “ribosome display”) does not rely on in vivo nucleic acid libraries, but it cannot identify activities other than binding activities and it is limited to the selection of proteins and cannot be used to select transcripts. Furthermore, to provide a link between the desired activity and the sequence that encodes it, ribosome display must maintain an actual physical linkage (bond) between the protein that exhibits the desired function and the nucleic acid that encodes the protein. RNA requires a bond between the protein and nucleic acid because the nucleic acids are sorted via the binding activity of the proteins. The present invention requires no physical bond because the sorting of the nucleic acids occurs before the functional assay. Therefore, any detectable property of the protein or the DNA or RNA can be assayed. In addition, in the context of screening, few laboratories can implement ribosome display successfully, in part because of the technical challenges of selection via the binding activity and because of the instability of the mRNA, which must be reverse transcribed.

Moreover, ribosome display can be used only with known genes because it is necessary to re-build the genes by replacing its stop codon with a coding sequence of several dozen nucleotides that are in phase with the coding sequence of the gene. Thus, prior art ribosome display can be used to select only the variants (obtained by mutagenesis or randomization of part of the sequence) of a known gene. Ribosome display cannot be used to find unknown genes that encode a given function.

Furthermore, although both ribosome display and the present invention use translation extract for in vitro expression, in ribosome display the extract is prepared only from specific mutant E. coli cells that lack the function responsible of ribosome dissociation. This mutation is known only in E. coli. In the present invention, the translation extract can be of the same or a similar origin as that of the sample.

2. Biological Genomic and cDNA Samples

As used herein, a “biological sample” is a nucleic acid-containing sample drawn directly from nature, directly from an organism or directly from portions or extracts of an organism. A biological sample, particularly a “crude” biological sample, often contains materials other than nucleic acids. Some examples of biological samples include soil, blood, water, tissue samples or biopsies, plant cuttings, and cultures (microbial, cellular or viral). A biological sample typically contains genomic or multiple types of nucleic acids, and either the existence, presence (in the sample), identity, properties or function of one or more of the nucleic acids is typically unknown to either the experimenter (i.e., the researcher using the invention) or unknown to both the experimenter and to science. The phrase “function of one or more of the nucleic acids” is shorthand for the function associated with the transcript or protein encoded by the one or more nucleic acids. In a preferred embodiment, the organisms in the biological sample are non-cultivable, which means they do not grow with currently known cultivation techniques. In another preferred embodiment, the method is performed without isolating the organism(s) in the biological sample from each other. In another preferred embodiment, the biological sample includes or derives from multiple types of cells or extracts or tissue samples from multiple organisms, and either the existence, presence (in the sample), identity, properties or function of at least one of the nucleic acids is unknown to the experimenter.

A “genomic sample” either contains non-coding and coding DNA or is drawn from a genome-wide library that contains the cDNA/mRNA representing all or virtually all of the genome of a species or subspecies of an organism. A genomic library is typically formed by isolating the native DNA from tissue or culture of the organism. Accordingly, a genomic sample often contains multiple types of nucleic acids, and either the existence, presence (in the sample), identity, properties or function of at least one of the nucleic acids is often unknown to either the experimenter (i.e., the researcher using the invention) or unknown to both the experimenter and to science. A genomic sample may or may not itself represent the whole genome of a species or subspecies. A “genome-wide sample” is genomic sample that, like a genome-wide library, does represent the whole genome of a species or subspecies.

As used herein, a “cDNA sample” is drawn from a cDNA library that contains cDNA/mRNA that does not represent the entire genome of a species or subspecies of an organism. Nevertheless, the existence, presence (in the sample), identity, properties or function of at least one of the nucleic acids in a cDNA sample is often unknown to the experimenter (i.e., the researcher using the invention) or even unknown to both the experimenter and to science.

Particularly promising is the possibility of using extremophilic enzymes in industrial processes (agribusiness, animal nutrition, paper, detergents, textile industries etc . . . ). An “extremophile” is a microorganism that can grow in extreme conditions such as those that are: acidophilic (e.g., pH lower than 2 as in coal deposits, sulfurous springs or areas of acid mine drainage); alkalinophilic (e.g., pH higher than 11 as in sewage sludge or alkaline lakes); psychrophilic (e.g., low temperatures at or below 0-4° C.); thermophilic (e.g., high temperatures at or above 60-70° C., like those in volcanos, deep ocean vents and geyser systems); barophilic (e.g., high pressure at the ocean bottom); halophilic (e.g., high salinity of solterns and saline lakes and seas such as the Dead Sea which has a salt concentration of 32 percent); radiophilic (e.g., nuclear waste sites); oligotrophic (few nutrients available); or anaerobic (without oxygen). Extremophilic bacteria, for example, can be contrasted with aerobic mesophilic bacteria, which live under normal temperature conditions at a pH around 7 and at about 1 atm pressure.

Accordingly, in a preferred embodiment of the invention the biological or genomic sample contains or is derived from one or more extremophiles or from a sample obtained in an extremophilic environment. In another preferred embodiment, the biological sample contains or is derived from one or more acidophiles, more preferably acidophiles that can grow in a pH lower than 2. In another preferred embodiment, the biological sample contains or is derived from one or more alkalinophiles, more preferably alkalinophiles that can grow in a pH higher than 11. In another preferred embodiment, the biological sample contains or is derived from one or more psychrophiles, more preferably psychrophiles that can grow in temperatures below 4° C. or even more preferably below 0° C. In another preferred embodiment, the biological sample contains or is derived from one or more thermophiles, more preferably thermophiles that can grow in temperatures above 60° C. or even more preferably above 70° C. In another preferred embodiment, the biological sample contains or is derived from one or more barophiles, more preferably those that can grow at those pressures at the bottom of the ocean. In another preferred embodiment, the biological sample contains or is derived from one or more halophiles, more preferably halophiles that can grow in a salt water concentration of approximately 25-32 percent salt. In another preferred embodiment, the biological sample contains or is derived from one or more radiophiles, more preferably those that can grow in or around nuclear waste. In another preferred embodiment, the biological sample contains or is derived from one or more oligotrophs. In yet another preferred embodiment, the biological sample contains or is derived from one or more anaerobes. In still another preferred embodiment, the biological sample contains or is derived from one or more extremophiles that fits into more than one of the foregoing subcategories of extremophile, such as thermophilic barophiles that grow in deep ocean thermal vents.

3. Nucleic Acids

A nucleic acid comprises one or more DNA, RNA or gene sequences, or fragments thereof. As intended herein, when a nucleic acid is obtained from a biological sample, genomic sample or cDNA sample, the existence, presence (in the sample), identity, properties or function of at least one nucleic acid is unknown to the experimenter or unknown to both the experimenter and to science.

In one embodiment, the existence of at least one of the nucleic acids is unknown to the experimenter. In another embodiment, the presence in the sample of at least one of the nucleic acids is unknown to the experimenter. In another embodiment, the identity of at least one of the nucleic acids in the sample is unknown to the experimenter. In another embodiment, the properties of at least one of the nucleic acids in the sample is unknown to the experimenter. In another embodiment, the function of at least one nucleic acid (i.e., the function of the transcript or protein encoded by the nucleic acid) in the sample is unknown to the experimenter. In a preferred embodiment, either the existence, presence, identity, properties or function of the majority of the nucleic acids in the sample is unknown to the experimenter. In another embodiment, either the existence, presence, identity, properties or function of all of the nucleic acids in the sample is unknown to the experimenter. In another embodiment, the existence of at least one of the nucleic acids is unknown to the experimenter and to science. In another embodiment, the presence in the sample of at least one of the nucleic acids is unknown to the experimenter and to science. In another embodiment, the identity of at least one of the nucleic acids in the sample is unknown to the experimenter and to science. In another embodiment, the properties of at least one of the nucleic acids in the sample is unknown to the experimenter and to science. In another embodiment, the function of at least one of the nucleic acids in the sample is unknown to the experimenter and to science. In a preferred embodiment, either the existence, presence, identity, properties or function of the majority of the nucleic acids in the sample is unknown to the experimenter and to science. In another embodiment, either the existence, presence, identity, properties or function of all of the nucleic acids in the sample is unknown to the experimenter and to science.

4. Selecting Desired Functions

In this step, the experimenter directly or indirectly chooses the particular function (or functions) that he is interested in before transcribing and optionally translating nucleic acids from the sample that may or may not encode that chosen function. In a preferred embodiment of the invention, selecting a desired function can additionally refer to directly or indirectly choosing the functions of interest before choosing or obtaining the biological or genomic sample from which the nucleic acids are obtained.

As used herein, “function” means a biological activity or affinity of the transcript or protein encoded by a nucleic acid. The transcript function could be, for example, a ribozyme, tRNA, Tm RNA or siRNA (small interfering RNA) activity or affinity. The protein function could, among other things, be a binding activity or an enzymatic activity such as an oxidoreductase, transferase, hydrolase, lyase, isomerase or ligase activity. When the desired function is enzymatic, the translated proteins can be tested for the function biochemically, for instance, by varying the conditions that affect enzymatic activity (e.g., pH, temperature, salt concentration) or by observing the kinetic (Vₘ, Kₘ) or inhibitory parameters (Kᵢ). When the desired function is an affinity, the proteins can be tested for it by determining their K_{d} or by determining which molecules have the most affinity for these proteins.

5. Preparing and Separating Nucleic Acids from Sample

These steps include preparing nucleic acids (i.e., DNA/RNA fragments) from the sample and then separating or sorting them out. Sorting out the prepared nucleic acids facilitates the link between the desired function and the corresponding encoding nucleic acid. Preparing the nucleic acids may comprise fragmentation (preferably random), extraction, purification and/or preparation of the ends for association with vector molecules. Fragmentation and extraction are used, for example, when the nucleic acids are from biological samples such as cells, viruses or blood. Advantageously, one or several endonucleases are applied to the nucleic acids of the sample or their PCR products. The nucleic acids can also be subjected to mechanical action, for example, by passage through a syringe needle, disruption under pressure or sonication. Where the biological sample comprises mRNA, the preparation may include a step of RT-PCR. Where the nucleic acids do not require fragmentation (e.g., nucleic acids from genomic libraries or cDNA libraries), these steps may still include extraction, purification and/or preparation of the ends for association with vector molecules.

When the sample comes from an eukaryote, the nucleic acid fragments obtained from the sample are preferably several dozens to several hundreds of kilobases in length. When the sample comes from a prokaryote, the nucleic acid fragments are preferably about 1 to several dozen kilobases in length, more preferably from about 1 to about 40 kb and still more preferably from about 1 to about 10 kb. Most preferably, such fragments are on the order of about 5 kb. In effect, the average size of a prokaryote gene is about 1 kb. By using fragments of 5 kb, the clones can carry the complete gene, including its proper ribosome-binding site. Alternatively, the fragments may carry only a partial or entire operon if it can encode the transcript or protein with the desired function.

Separating (or “sorting” or “isolating”) the prepared nucleic acids can be accomplished by various means including: through extreme dilution, by tagging the nucleic acids with a label (such as streptavidine, biotin, a polypyrol group or an antibody), or by cloning the nucleic acids with a DNA plasmid vector. In the invention, separating the nucleic acids preferably entails inserting the nucleic acids into vector molecules comprising one or several polynucleotide sequences with at least one transcription promoter, thereby forming recombinant vector molecules. In a preferred embodiment, the vector molecule comprises two polynucleotide sequences each with a different transcription promoter. In this embodiment, each sequence is associated with an end of one of the fragments. In a more preferred embodiment, the transcription promoter carried by the vector molecule is more preferably a strong-type promoter such as an RNA polymerase transcription promoter of the T7, SP6, Qβ or λ phage, most preferably T7 RNA polymerase. Preferably, the vector molecules also comprise a substance that facilitates isolation of the nucleic acid fragments, such as streptavidine, biotin, polypyrol groups or antibodies. When the sample comes from a cDNA library, the vector molecule preferably further comprises a translation initiation sequence corresponding to the translation extract used at the translation step of the method embodiments of the invention that both transcribe and translate the nucleic acids.

When the vector molecule is plasmidic, such as pBR322 or pACYC184, the nucleic acid fragments are inserted in the vector molecules at a cloning site or via a restriction cassette. Preferably, this plasmidic vector comprises an RNA polymerase promoter at one side of the cloning site and optionally an RNA polymerase terminator at the other side. It is also possible to design a vector comprising a cloning site surrounded by two different or identical RNA polymerase promoters and possibly flanked on both sides by a corresponding RNA polymerase terminator or terminators. Preferably, the plasmidic vector does not permit or facilitate in vivo expression of the inserted nucleic acid fragment. In other words, its promoters and optionally its terminators do not function in the microorganism used to isolate the recombinant vectors.

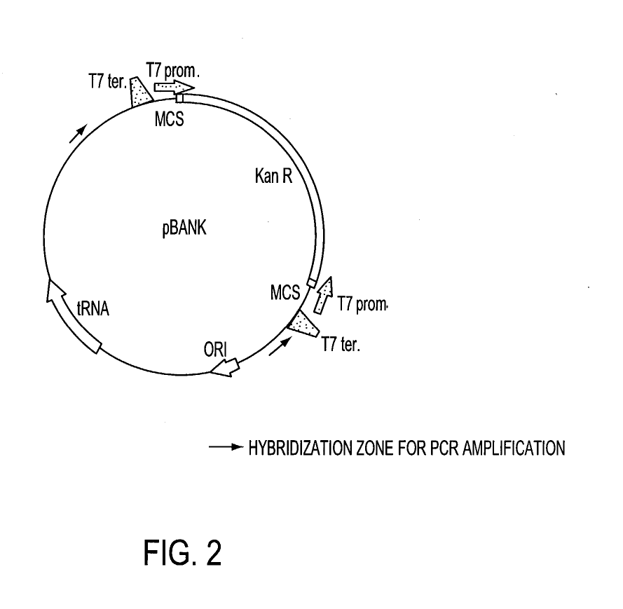

In certain embodiments of the invention, the nucleic acid fragments are cloned via the plasmid vector. An example of a suitable vector is represented in FIG. 2, which may be constructed as follows:

A plasmidic replication origin.

A cloning site surrounded by two identical promoters, such as that of the T7 RNA polymerase, or any other strong RNA polymerase promoter, such as Qβ, T3, SP6, etc., and optionally flanked on both sides by the same RNA polymerase terminator. These promoters and terminator, if it is present, preferably do not function in the microorganism used to separate the recombinant vectors. Such a construction permits in vitro transcription of a DNA fragment inserted in the cloning site, regardless of its sense of insertion. The probability of finding a good clone is therefore multiplied by two. The average size of a prokaryotic gene is about 1000 bp. By using prokaryotic DNA fragments of about 5000 bp to generate the library, it is highly probable that clones carrying the complete genes will be obtained, with their proper ribosome binding site (or RBS for Ribosome Binding Site). With this double promoter system, the gene is located in the worst case 2000 bases from the beginning of the mRNA, which permits effective expression of the corresponding protein by the process of the invention (as reported in the experiment hereinafter on the Beta-lactamase activity).

Optionally, some specific sequences on both sides of the terminators can be used as hybridization sites for a PCR amplification of the nucleic acid fragment carried by the vector.

A selection gene composed of a tRNA gene (4). Optionally, in parallel, an antibiotic resistance gene (or another type of selection gene) is inserted in the cloning site. This antibiotic selection is used only for the preparative amplification of the cloning vector. In effect, during the insertion of each of the nucleic acids into a vector, a DNA fragment is substituted for this resistance gene. This system has the advantage of not depending on an antibiotic selection, which raises problems of contamination and degradation of the antibiotic, and permits obtaining a recombinant vector not possessing an ORF other than that possibly introduced by the heterologous fragment. On the other hand, it permits a very rapid evaluation of the level of negative clones, by practicing a parallel spreading of a fraction of the library on minimum medium and on a medium containing the selection antibiotic.

When the vector molecules are plasmidic vectors, they can be isolated by transforming host cells with the entirety of the recombinant vectors to create a library of clones and then by extracting each clone by any appropriate means, such as by plasmid miniprep and possibly digestion or by PCR. Preferably, extracting the clones of the nucleic acids employs PCR with oligonucleotides protected by phosphorothioate groups from 5′ nuclease attacks by the nucleases contained in the translation medium.

Isolating or separating the nucleic acids may include creating a genomic library from the DNA in a biological sample. Genomic DNA can be isolated from the biological sample using QIAamp DNA MINI KIT (QIAGEN). A plasmid library can be constructed by ligation of genomic DNA, which was previously partially digested by Sau 3A I, into the Bam HI site of a plasmidic vector such as pBSKS (Stratagene). The plasmid library is amplified by transformation of E. coli MC1061 strain. Isolated E. coli colonies are individually automatically selected (for example, by a Flexis colony picker) and used to inoculate culture plates containing 150 μl of Luria Broth with 100 mg/L ampicillin per well. Cultures are grown overnight at 37° C. Each well contains a plasmidic vector having a fragment of genomic DNA. Each culture undergoes a lysis step to recover this plasmidic vector having a fragment of genomic DNA.

6. Transcribing the Prepared Nucleic Acids

The transcription is done as described in Pokrovskaya, I. D. and Gurevich, V. V. (Analyt. Biochem. 1994. 220, 420-423). When the sample is drawn from eukaryotes, the step of treating the nucleic acid-vectors in vitro to generate transcripts comprises, for example, in vitro splicing and maturation reactions of the mRNA in a nuclear extract (3).

7. Translating the Transcripts

This step is performed only in the transcription-translation embodiments of the invention. The translation is done as described in the articles of Pratt J. M. 1984 or of Zubay G., 1973. In one embodiment, the translation step is coupled with the transcription step and can be simultaneous, i.e., transcription and translation may occur at the same time in the same test tube. Alternatively, transcription and translation may be divided into two temporally distinct steps. The division into two temporally distinct steps permits optimization of the yields of each step. Thus, production of greater quantities of proteins may be obtained. This is particularly advantageous in the case of enzymes with weak specific activity. The division of these steps also allows normalization of the translation and enables later comparison of different expressed functions. When a DNA template was prepared by PCR, division of transcription and translation further avoids degradation of the DNA template by nucleases. In effect, the components of the transcription reaction are less contaminated by the nucleases than the components of the translation extracts.

The division of transcription and translation into distinct steps also allows use of different translation extracts depending on the origin of the screened DNA. However, translation is advantageously carried out with a translation extract of the same origin or of a close origin to that of the biological sample. As such, the correspondence between the origin of the transcript translation signals and the cellular extract is optimized for translation effectiveness.

A translation extract of eukaryotic cells can be used to screen a eukaryotic DNA library. Preferably, these extracts either do not themselves inherently contain nucleic acids that encode the desired function or, if they do, the conditions in which the invention operates are adjusted so that the nucleic acids of the extracts do not encode detectable amounts during the method. For example, if the experimenter selects a thermophilic beta-galactosidase function, then using a translation extract containing a nucleic acid that encodes a mesophilic beta-galactosidase activity will not cause a problem if the translation step or testing step occurs at a high temperature suitable only for thermophiles.

The invention facilitates a correspondence between the translation extract that is used and the expression punctuation of the transcripts, e.g., the types of start and stop signals they have. Although the genetic code is nearly universal, efficiency of translation varies greatly depending on the type of extract used to translate a given gene. The composition of a translation extract reflects its origin and each extract is better suited to translate homologous genes from organisms that have similar characteristics as the sample organisms. One such characteristic is expression punctuation. For example, eukaryotic extracts cannot translate genes belonging to an operon, and the ability of prokaryotic extracts to translate genes belonging to an operon depends to an extent on the punctuation within the operon.

Another embodiment of the invention comprises using a mixture of several translation extracts. For example, the mixture could contain a translation extract of E. coli over-expressing a chaperon A protein and a translation extract of E. coli over-expressing a chaperon B protein. The translation extract could also contain one or several tRNAs specific for a particular codon. For example, a translation extract would permit translation of an mRNA containing an amber codon if the translation extract included the right tRNA suppressor. In another embodiment, the invention comprises adding to the translation extract a substance that favors refolding or maturation of the expressed proteins, for example, chaperons, detergents, sulfobetaines, membrane extracts, etc. Translation can also be carried out with a universal translation extract regardless of the origin of the sample. For example, a translation extract from E. coli can be supplemented with the foregoing substances (tRNA, chaperon, etc.).

8. Testing for Desired Function

The specificity of the test is chosen to reveal the desired function that is to expose the biological activity of the transcripts or of the proteins.

In the transcription-translation embodiments of the invention, testing preferably includes screening for expressed proteins, for example, using, ELISA. Thus, the separated nucleic acids that do not possess an ORF on their insert can be eliminated. The actual test for the desired function can be carried out by various means for detecting a protein with a desired function. For example, enzymes can be detected using fluorimetry, colorimetry, absorbance, viscosity and the so forth. Testing for a desired affinity may entail, for example, using double stranded DNA binding proteins, radio-labeled ligands bound to receptors, or certain antibody-antigen complexes. Examples of useful antibody-antigen complexes include those formed by: immobilizing antigens followed by binding them to desired antibodies followed by binding the antibodies to anti-antibodies coupled to a signal; and binding immobilized goat antibodies to specific antigens that can be detected by a rabbit antibody (sandwich formation) indirectly coupled or not to a reporter (alkaline phosphatase or peroxidase type).

In preferred embodiments, particularly those where enzymatic functions or affinities are desired, the invention not only identifies nucleic acids that encode the desired functions, it also quantifies the degree to which they encode the desired functions. In other embodiments, the invention also characterizes or determines optimal conditions for eliciting the desired function, such as optimal temperature, pH, and salinity. In other embodiments, the invention also characterizes various properties of the nucleic acids or the proteins they encode, such as molecular weight, sequence of residues or inhibition conditions of its activity.

In transcription-only embodiments, the transcript is tested for a desired function like an ribozyme, tRNA, Tm RNA or SI RNA (small interferring RNA) function. The invention can test for a ribozyme with an endonuclease activity, for example, by using a nucleotide matrix with a fluorescent group at one end and a “quencher” group at the other. When the matrix is cut by the ribozyme, the fluorescent group is freed from proximity to the quencher group, thereby providing a detectable signal.

The invention can test for a desired tRNA (using a fraction of the reaction mixture potentially containing the tRNA) by putting the tRNA in an in vitro translation reaction containing a reporter gene with a codon that only the tRNA can read. If the activity of the reporter protein is detected, the presence of the tRNA in the initial fraction is thereby detected, because such tRNA is necessary for the in vitro translation of the reporter gene.

In preferred embodiments, the invention further includes identifying at least one nucleic acid that encodes either the protein or the transcript associated with or exhibiting the function initially selected by the experimenter. The identification is achieved without sequencing due to the link between the protein or transcript and the corresponding nucleic acid provided by the invention.

9. Apparatus, Device and Kit Embodiments

The methods of the invention can be carried out in the apparatus, devices and kits of the invention. Preferably, the methods of the invention are entirely carried out on a solid chip-type support or a membrane or a nanotitration plate. The chip-type support can be a glass plate, a nitrocellulose membrane or any other support known to a person skilled in the art. The nucleic acids associated with the vector molecule are isolated on this chip-type or nanotitration plate support, and the reactants permitting the implementation of the process of the invention are deposited on this support. The test for the desired function can be directly conducted on the support after a possible washing of the latter. When the vector molecule is plasmidic or the methods of the invention are carried out on a support, the colonies transformed by the recombinant vectors are transferred separately from the others on a same support, then lysed in situ (3) such that each colony can liberate on the support the copies of the recombinant vector that it contains. Another embodiment comprises separately loading on a same support each recombinant vector or part of it. It is thus possible to deposit reactants permitting the carrying out of an in vitro protein expression reaction on the support having the deposited DNA. The test for the function can be conducted directly on the support, preferably after washing the support.

Another embodiment invention comprises an automated device for implementing the methods of the invention. Preferably, the device comprises a layout of one or several supports, robots, automatic machines and readers. When the vector molecule is plasmidic, automation can be achieved via the following:

Each recombinant vector of the library formed at step (c) can be put in culture on a support, in a microplate well by a Colony Picker type robot.

This culture can be used for a plasmidic extraction step carried out by a Biorobot 9600 (QIAGEN) type robot, or for a PCR amplification step implemented by a MultiProbe type machine (PACKARD) on a PTC 200 or PTC 225 (automated lid-MJ RESEARCH) type automatic thermocyler.

The optional purification of the PCR products can be conducted by the BioRobot 9600 automatic machine.

The in vitro protein expression reaction of steps (e) and (f) can be directed entirely by the MultiProbe robot. The tests of the functions of the transcripts obtained at step (e) can be effected on the robot pipetor and the reading of the results is obtained on a corresponding reader. If the transcription reaction is separated from the translation reaction, the optional purification of the mRNA can be carried out by the BioRobot 9600.

The tests of the activity of the proteins synthesized at step (f) are carried out by the robot pipetor, and the reading of the results is obtained on the reader (spectrophotometry, colorimetry, fluorimetry, etc., according to the test carried out) of micro plaques or by any other appropriate means.

### EXAMPLE 1

### I. Materials

1) Strains and Plasmids

The vector pET26b+ is part of the family of pET vectors developed by Studier and Moffatt (8) and commercialized by the NOVAGEN Corporation. This vector permits expression of the genes under the control of the T7 phage promoter. The PINPOINT™ (PROMEGA) vector carries the cat gene (coding for the chloramphenicol acetyltransferase) under the control of the T7 phage RNA polymerase. The vector pHS2-22-21 was constructed by introducing by reverse PCR the cutting site recognized by the intein 2 of the Tfu DNA polymerase positioned in the polycloning site of the plasmid pUC19. The vector pHS2-22-21 corresponds to pUC 19 containing the homing site (43 bp) of the intein or the site in which the intein gene is inserted.

The strain XL1-Blue [Tn10 proA⁺B⁺lacl^{q} Δ (lacZ)M15/recA1 endA1 gyrA96 (Nal^{r}) thi bsdR17 (rₖmₖ⁺)supE44 relA1 lac) was used for the amplification of the plasmidic DNA.

2) Reagents

Table I shows the restriction and modification enzymes used for Example I.

TABLE I

| Enzyme | Concentration | Supplier |
|---|---|---|
| Sca I | 10 U/μl | Appligene Oncor |
| Sal I | 8 U/μl | New England Biolabs |
| Nde I | 20 U/μl | New England Biolabs |
| Barn HI | 20 U/μl | New England Biolabs |
| Eco RI | 20 U/μl | New England Biolabs |
| T7 RNA | 50 U/μl | New England Biolabs |
| polymerase | | |
| T4 DNA ligase | 400 U/μl | New England Biolabs |

Table II shows the buffers used for Example I.

TABLE II

| Buffers | Composition |
|---|---|
| T | Tris HCl 10 mM pH 8.0 |
| T7 RNA polymerase | Tris HCl 400 mM pH 7.9, 60 mM MgCl₂, 20 |
| (10 X) | MM spermidine, 10 mM DTT |
| IT2 (10X) | Tris-Oac 500 mM pH 8.0, 750 mM |
| | Mg(Oac)₂, 100 mM NH₄OAc |
| TADH (2X) | Glycine 100 mM (NaOH pH 10), 20 mM |
| | Butanol 1, 0.9 M NaCl, 4 mM NADP |
| LIP | KH₂PO₄ 0.1 M, pH 6.8, dioxane 5%, |
| | Thesit 5% |
| BETA | NaP 50 mM pH 7.0, 100 μg/ml |
| | Nitrocephine, 0.25 mM DMSO |
| Ligation 10X | Tris HCl 500 mM pH 7.5, 100 mM MgCl₂, |
| | 100 Mm DTT, 10 mM ATP, 250 μg/ml BSA |

### II. Preparation and Test of the Plasmids

1) Construction

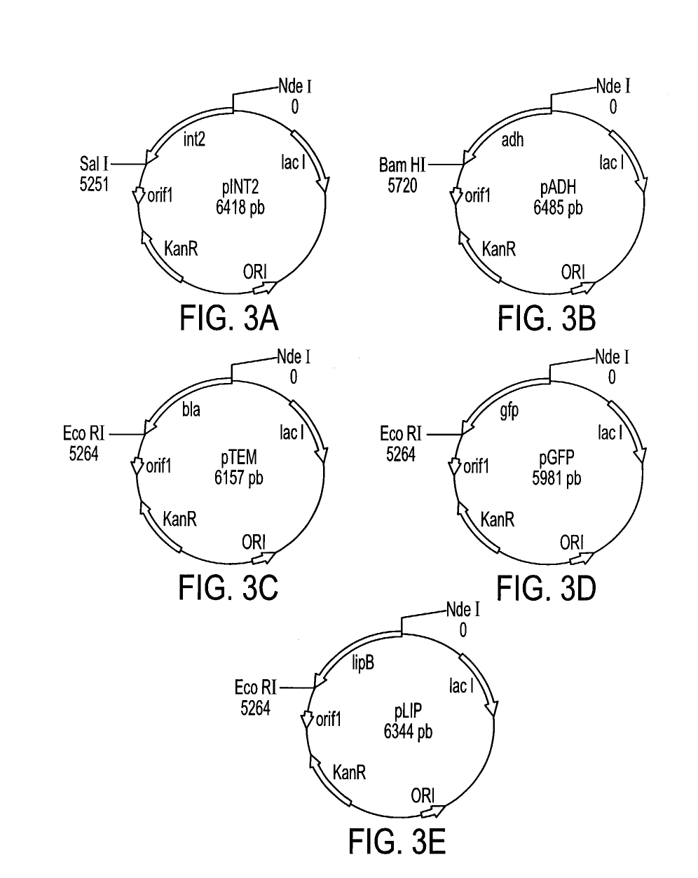

The gene of the intein 2 of the Tfu DNA polymerase (itfu2) (Accession number in gene library: Z69822) was inserted between the restriction sites Nde I and Sal 1 of the vector pET26b+ in order to create the plasmid pET26-Tfu2 represented in FIG. 3. In similar fashion, the alcohol dehydrogenase gene (adh) of Thermococcus hydrothermalis was inserted between the restriction sites Nde I and Bam HI of the vector pET26B+ in order to create the vector pADH, and the genes of the Beta-lactamase TEM-1 (bla) of Escherichia coli (9), and of the Green Fluorescent Protein (gfp) of Aequorea Victoria (6) and of the lipase B of Candida Antarctica (lipB) (Accession number Y14015 in gene library) were inserted respectively between the restriction sites Nde I and Eco RI of the vector pET26b+, in order to create the plasmids pADH, pTEM, pGFP and pLIP represented in FIG. 3. For each one of these four genes, the restriction site Nde I is over located with the codon ATG of the translation initiation.

Each construction was verified by several restriction profile analyses. 200 μl of XL1-Blue chimiocompetent cells (1) were transformed with 10 ng of each plasmid by a thermal shock (2), and the cells thus transformed were spread out on a solid LB medium containing 60 μg/ml of kanamycin and 12.5 μg/ml of tetracycline. Starting from a clone of each one of these transformations, a plasmidic DNA maxipreparation was carried out with a TIP100 type column (QIAGEN). After precipitation in isopropanol, each plasmidic DNA sample was resuspended in 100 μl of buffer T. The concentration of these plasmidic DNAs was evaluated by a spectrophotometric measurement at 260 nm. The purity of each plasmidic DNA was verified by depositing 0.2 μl of each of the vectors on agarose gel TBE 1%.

2) In Vivo Expression Tests—Activity Tests

pTEM: 200 μl of BL21 DE3 (pLysS) chimiocompetent cells were transformed with 10 ng of the pTEM plasmid by a thermal shock, and the cells thus transformed were spread out on a solid LB medium containing 60 μg/ml of kanamycin, 20 μg/ml of chloramphenicol, 32 μg/ml of IPTG and 100 μg/ml of ampicillin. After incubation one night at 37° C., numerous colonies could be observed on the Petri dish, thereby revealing that the TEM-1 gene of the plasmid pTEM is expressed and functional, and that it confers ampicillin resistance.

pGFP: 200 μl of BL21 DE3 (pLysS) chimiocompetent cells were transformed with 10 ng of the plasmid pGFP by a thermal shock, and the cells thereby transformed were spread out on a solid LB medium containing 60 μg/ml of kanamycin, 20 μg/ml of chloramphenicol and 32 μg/ml of IPTG. After incubation one night at 37° C., numerous colonies could be observed on the Petri dish. All of these reacted to ultraviolet excitation (at about 400 nm) by emitting a green fluorescent light, which permitted verification that the b gene of the plasmid pGFP is expressed and functional.

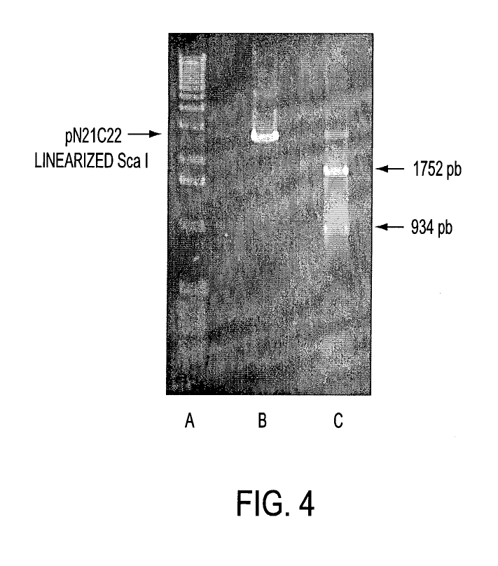

pET26-Tfu2: 200 μl of Bl21 DE3 (pLysS) chimiocompetent cells were transformed with 10 ng of the plasmid pET26-Tfu2 by a thermal shock, and the cells transformed thereby were spread out on a solid LB medium containing 60 μg/ml of kanamycin and 20 μg/ml of chloramphenicol. A culture carried out starting from a clone of this transformation was induced at DO₆₀₀ₙₘ=0.5 with 0.5 mM of IPTG for two hours at 37° C. After centrifugation, the bacterial precipitate was resuspended in 20 mM sodium phosphate buffer pH 7.5, and a cellular lysate was obtained after several cycles of freezing/thawing. The intein 2 was then purified on a Qfast-Flow column with a NaCl gradient. The activity of this enzyme was tested according to the following protocol: 1 μl of the elution fraction having the highest pure enzyme concentration was diluted one hundred times. One μl of this dilution was incubated 15 minutes at 70° C. with 3 μl of IT2 buffer and 220 ng of the pHS2-22-21 plasmid, linearized with the restriction enzyme Sca I, in a final volume of 30 μl. 15 μl of this digestion mixture were deposited on an agarose gel TBE 1%. After migration and staining with ethidium bromide, the gel was exposed to ultraviolet. As shown in FIG. 4, the analysis of this gel reveals the presences of two bands of respectively 934 bp and 1752 bp, corresponding to the cutting of the pHS2-22-21 vector (Sca I linearized) by the intein 2. The gene itfu2 of the plasmid pET26-Tfu2 is therefore expressed and its product, the intein 2, is active.

pADH: 200 μl of BL21 DE3 (pLysS) chimiocompetent cells were transformed with 10 ng of the pADH plasmid by a thermal shock, and the cells thus transformed were spread out on a solid LB medium containing 60 μg/ml of kanamycin and 20 μg/ml of chloramphenicol. A culture carried out starting from a clone of this transformation was induced at DO₆₀₀ₙₘ=0.6 with 1 mM of IPTG for three hours at 37° C. After centrifugation, the bacterial precipitate was taken up in a sodium phosphate buffer 50 mM-MgCl₂ 10 mM pH 8.0, and the cellular lysate was obtained by incubating 30 minutes over ice in the presence of 1 μg/ml of lysozyme, 10 μg/ml of RNAse A and 100 μg/ml of DNAse I. The centrifugation supernatant of this extraction step was incubated 30 minutes at 50° C., and centrifuged again. The supernatant of this last step was used as an enzymatic extract for the measurements of activity. A negative control was made up in parallel by carrying out a similar extraction on a culture of BL21 DE3 (pLysS) cells excluding plasmid. The alcohol dehydrogenase activity was tested by following with a spectrophotometer the reduction kinetics of the NADP to NADPH at 340 nm. For this, 10 μl of the enzymatic extract, or of the control, were incubated at 50° C. for five minutes with 500 μl of TADH buffer and 490 μl of water. Under these conditions, an activity of 15.6 UDO/min/ml of enzymatic extract was detected, against 0 UDO/min/ml for the control. The pADH plasmid gene adh is therefore expressed and its product, the alcohol dehydrogenase, is active.

### III. Protein Expression Trials In Vitro with a Translation Extract Prepared from Mesophilic Strains (37° C.)

4 μg of each vector were precipitated in the presence of one tenth volume of sodium acetate 3 M pH 6.0, and two volumes of absolute alcohol. The precipitates were rinsed with 70% ethanol in order to eliminate any trace of salts. Each precipitated DNA was resuspended in 4 μl of buffer T.

This DNA is incubated two hours at 37° C. in a protein expression mixture in vitro containing 0.1 mM of each one of the 20 amino acids, 20 μl of “S30 Premix” extract and 15 μl of “T7 S30 extract” (PROMEGA) in a final volume of 50 μl. The “S30 Premix” and “T7 S30 extract” extracts contain all the elements necessary for an in vitro transcription reaction coupled with a translation reaction, notably: T7 RNA polymerase, CTP, UTP, GTP, ATP, tRNAs, EDTA, folic acid and appropriate salts. The translation extract was produced according to the procedure described by Zubay (10) from an Escherichia coli B strain deficient in endoproteinase OmpT and in Ion protease, which better stabilizes the proteins expressed in vitro.

A negative control was prepared by incubating ultra pure sterile water in place of the DNA in the transcription-translation mixture. The positive control was formed by incubating 2 μg of PINPOINT™ plasmid.

The in vitro protein expression reactions were preserved on ice until the enzymatic activity of each sample could be evaluated.

### IV Measurement of the Activities

1) Mesophilic Enzymes

a) GFP Activity

FIG. 5 shows the tube containing the product of the in vitro protein expression reaction with the pGFP vector that was exposed to ultraviolet at about 400 nm, beside the tube containing the control reaction. Only the tube containing the in vitro protein expression reaction of the pGFP plasmid emitted a green fluorescent light. The in vitro protein expression reaction therefore permits the production of a GFP protein having a fluorescent activity.

b) Beta-Lactamase Activity.

The Beta-lactamase activity was evaluated by following by spectrophotometry the degradation kinetics of nitrocephine, a chromogenic cephalosporin, at 486 nm (5). For this, 5, 10, or 20 μl of the in vitro protein expression reaction with the pTEM vector are incubated 2 minutes at 37° C. in a BETA buffer, final volume 1 ml. The average activity of the in vitro protein expression reaction with the pTEM vector could be estimated at 8.9 UDO/min/ml of extract, against 0.6 UDO/min/ml of extract with the control in vitro protein expression reaction. The protein expression reaction in vitro therefore permitted the synthesis of an active Beta-lactamase, capable of degrading the nitrocephine in vitro.

The control PINPOINT™ vector carries the bla gene under the control of its promoter. However, the Beta-lactamase activity of the in vitro protein expression reaction with this vector was tested and evaluated at 6 UDO/min/ml of extract. It is useful to note that the addition of rifampicine at 1 ng/μl the RNA polymerase inhibitor of E. coli, does not significantly modify the in vitro expression of the Beta-lactamase with the PINPOINT™ vector. The bla gene of the PINPOINT™ vector, located 2123 bp downstream of the T7promoter is therefore transcribed and translated effectively. This implies that a gene located 2000 bp downstream of a T7 promoter is effectively transcribed and translated during an in vitro protein expression reaction.

2) Thermophilic Enzymes

a) Intein 2 Activity.

The in vitro protein expression reaction with the pET26-Tfu2 vector was tested in order to know if an active intein 2 could be produced. For this, 5 μl of this in vitro protein expression reaction were incubated 20 minutes at 70° C. with 220 ng of pHS2-22-21 vector, linearized with Sca I, and 3 μl of IT2 buffer in a final volume of 30 μl. A negative control was formed by replacing the 5 μl of the in vitro protein expression reaction with water. The positive control contained 1 μl of the purified intein 2 fraction produced in vitro diluted to 1/100^{th}. Finally, a specificity control was made by incubating 5 μl of the in vitro protein expression reaction not having received any DNA.

After incubation, the four trials underwent a phenol-chloroform extraction and an ethanol precipitation, followed by rinsing with 70% ethanol in order to eliminate salts. Each precipitate was taken up in 10 μl of buffer T, and 8 μl were deposited on an agarose TBE 1% gel. After migration and staining with ethidium bromide, the gel was exposed to ultraviolet in order to analyze the restriction profiles. The appearance of bands at 934 and 1752 bp on track 5 of FIG. 6, identical to the bands of track 4 (positive control) reveals that the intein 2 is indeed produced by the in vitro protein expression reaction, and that this enzyme is active. In addition, the in vitro protein expression reaction is specific since no other digestion band can be observed on the control of track 3.

Alcohol Dehydrogenase Activity

Using spectrophotometry, the alcohol dehydrogenase activity was tested by following the reduction kinetics from NADP to NADPH at 340 nm. For this, 5, 10, or 15 μl of the in vitro protein expression reaction with the vector pADH, or without DNA, were incubated at 50° C. for five minutes with 500 μl of TADH buffer in a final volume of 1 ml. Under these conditions, the average activity of the in vitro protein expression reaction with the pADH vector was estimated at 2.3 UDO/min/ml of extract, against 0.32 UDO/min/ml of extract for the control (in vitro protein expression reaction with water). The ADH of Thermococcus hydrothermalis (accession number Y14015 in gene library) was therefore produced in an active form during the in vitro protein expression reaction.

3) Psychrophilic Enzyme

The lipase activity was tested by following, the spectrophotometric degradation kinetics of a chromogenic lipid (1,2-O-dilauryl-rac-glycero-3-glutaric acid-resorufin ester) at 572 nm. For this, 5 μl of the in vitro protein expression reaction with the pLIP vector, or without DNA were incubated at 70° C. for fifteen minutes in the reaction buffer in the presence of 100 μg of substrate. Under these conditions, the activity of the in vitro protein expression reaction with the pLIP vector was estimated at 0.50 UDO/min/ml of extract against 0.04 UDO/min/ml of extract for the control. The lipase B of Candida Antarctica (Eukaryotic organism) was therefore produced in active form during the in vitro protein expression reaction.

### V. Using a Translation Extract Containing a Mesophilic Activity for Detecting a Thermophilic Property

The beta-galactosidase gene of Thermotoga neapolitana was inserted in a vector containing the T7 RNA polymerase transcription promoter. The vector thus obtained was used for carrying out an in vitro protein expression reaction with a translation extract of an E. coli strain possessing a beta-galactosidase activity. In parallel, an in vitro protein expression reaction without DNA was carried out. By incubating a fraction of each one of the in vitro protein expression reactions at 37° C. in the presence of Xgal at 37° C. in a sodium phosphate buffer (50 mM, pH 7), the two tubes changed to a blue color in minutes (cf FIG. 7 tubes A and B). By incubating a fraction of each one of the in vitro protein expression reactions in the presence of Xgal under the same conditions but at 70° C., only the tube corresponding to the in vitro protein expression reaction with the plasmid coding for the thermophilic beta-galactosidase changed to the blue color pellet (cf FIG. 7 tubes C and D: these two tubes were centrifuged in order to color the proteins of the mesophilic translation extract which precipitated as a consequence of their thermic denaturation during the activity test at 70° C.). The mesophilic beta-galactosidase activity is no longer detectable at this temperature (thermal denaturation of the mesophilic beta-galactosidase). It is therefore possible to use a translation extract containing a property similar to the sought-after property if this property is undectable under the detection conditions of the sought-after property.

### VI. Translation Extract Prepared from Extremophilic Organisms

Translation extracts of other organisms, and in particular of extremophilic organisms, can be prepared starting from cells according to one of the procedures described by Zubay (1973) (10) or by Pratt (1984) (7). The speed of centrifugation, the conditions of cell breakage, and the different reaction or preparation buffers will be adjusted for each type of translation extract by systematic trials. By thus practicing the range of translation extracts, it becomes possible to translate a gene regardless of its genetic origin. The invention thereby facilitates a correspondence between the translation system and the punctuation of expression of the gene encoding the desired function.

### EXAMPLE 2

### Title: Translation of mRNA of Genomic Fragment of a Thermophilic Bacteria (Length 5 kb) Containing an Esterase Gene in the in Vitro Cell-Free Translation System

I. In Vitro Translation Sytem Based on the Pyrococcus horikoshii Extract.

Transcription of genomic fragment placed under the T7 promoter in the pBSKS vector was done as described in Pokrovskaya, I. D. and Gurevich, V. V. (Analyt. Biochem. 1994. 220, 420-423). mRNA was purified via P6 gel-filtration column (BioRad). T7 RNA polymerase used for transcription was from (Hybaid).

The standard translation reaction mixture of 80 microliters contains 0.3 volumes of S30 Pyrcoccus horikoshii extract prepared as described by Zubay (1973) and 5 microliters of purified transcription mixture or 0,4 mg/ml purified RNA. The translation extract is supplemented as described by Kigawa et al. (FEBS Lett. 1999; 442(1):15-9) with small modifications. The standard translation reaction mixture contains 80 mM of acetyl phosphate instead of 80 mM creatine phosphate and 0,25 mg/ml creatine phosphokinase and, in addition, 2,5 mM spermine. MgAc2 was also added in concentrations of 12, 14, 16, 18 mM. Reactions were conducted for 3 h in a water bath set at 65° C.

10 ml of the reaction mixture was used for testing a function using a C10 substrate (CIO or 2-hydroxy-4-(p-nitrophenoxy)-butyldecanoate substrate described in Lagarde et al., Org. Process Res. Dev. 2002; 6(4); 441-445). Incubation with the C10 substrate was done at 95° C. during 40 min.

II. In Vitro Translation System Based on E. coli S30 Extract.

Transcription of genomic fragment placed under the T7 promoter in the pBSKS vector was done as described in Pokrovskaya, I. D. and Gurevich, V. V. (Analyt. Biochem. 1994. 220, 420-423). mRNA was purified via P6 gel-filtration column (BioRad). T7 RNA polymerase used for transcription was from (Hybaid).

The standard reaction mixture of 80 microliters contains 0.3 volumes of S30 E. coli extract prepared as described in Zubay (1973) and 5 microliters of purified transcription mixture or 0,4 mg/ml purified RNA. The translation extract is supplemented as described by Kigawa et al. (FEBS Lett. 1999 Jan. 8;442(1):15-9) with small modifications. The standard translation reaction mixture contains 80 mM of acetyl phosphate instead of 80 mM creatine phosphate and 0,25 mg/ml creatine phosphokinase. MgAc2 was also added in concentrations of 10, 12, 14, 16 mM. Reactions were conducted for 3 h in a water bath set at 37° C.

10 ml of the cell-free translation reaction mix was used for testing the function using C10 substrate (C10 or 2-hydroxy-4-(p-nitrophenoxy)-butyldecanoate substrate described in Lagarde et al., Org. Process Res. Dev. 2002; 6(4); 441-445). Incubation with the C10 substrate was done at 95° C. during 40 min.

III. Results

Table III hereunder reports the enhanced translational level of genomic fragment RNA from Hyperthermophilic Archaeon in homologous Pyrococcus horikoshii extract compare to that in heterologous E. coli extract. Esterase functional activity is two fold higher by using thermophilic Pyrycoccus horikoshii extract based translation cell-free systems programmed with 5 kb long RNA. Esterase expression is shown in OD414 units.

These two examples show preferred cell free extracts facilitating detection of a function encoded by a genomic fragment. Preferred cell free extracts share a close phylogenetic origin with the genomic DNA that is screened, e.g., a thermophilic extract is preferably used to screen genomic DNA from a thermophilic bacteria.

TABLE III

Functional activity of
synthesized esterase, OD₄₁₄

| MgAc₂, mM | 10 | 12 | 14 | 16 | 18 | 20 |
|---|---|---|---|---|---|---|
| Pyrococcus horikoshii | nd | nd | 0.01 | 0,03 | 0.147 | 0,221 |
| E. coli | 0,094 | 0,093 | 0,072 | 0,05 | nd | nd |

FIG. 8 shows detection of an esterase function using a Pyrococcus horikoshii cell free extract. FIG. 9 shows detection of an esterase function using an E. coli cell free extract.

### BIBLIOGRAPHIC REFERENCES

1) Hanahan D., (1985), Techniques for transformation of Excherichia coli, in DNA cloning: a practical approach. Glover D. M. (Ed.), IRL Press, Oxford vol. I, 109-135.

2) Maniatis T., Fristch E. F. and Sambrook J., (1982), Molecular cloning. A laboratory Manual, Cold Spring Harbor Laboratory, Cold Spring Harbor, N.Y.

3) Masson J. M., Meuric P., Grunstein M., Abelson J. and Miller H. J., (1987), Expression of a set of synthetic suppressor tRNA^{Phe} genes in Saccharomyces cerevisiae, Proc. Natl. Acad. Sci. UAS, 84, 6815-6819.

4) Normanly J., Masson J. M., Kleina L. G., Abelson J. and Miller J. H., 1986, Construction of two Escherichia coli amber suppressor genes: tRNA^{Phe}CUA and tRNA^{CYS}CUA, Proc. Natl. Acad. Sci. USA, 83, 6548-6552.

5) O'Callaghan C. H., Morris A., Kiby S. M. and Shingler A. H., 1972, Novel method for detection of beta-lactamase by using a chromogenic cephalosporin substrate, Antimicrob. Agents Chemother., 1, 283-288.

6) Prasher D. C., Eckenrode V. K., Ward W. W., Prendergast F. G. and Cormier M. J., 1992 Primary structure of the Aequorea Victoria green-fluorescent protein, Gene, 111, 229-233.

7) Pratt J. M., 1984, Coupled transcription-translation in prokaryotic cell-free systems, Transcription and Translation: A practical approach, Hames B. D. and Higgins S. J. Eds, JRL Press.

8) Studier F. W. and Moffatt B. A., 1986, Use of bacteriophage T7 RNA polymerase to direct selective high-level expression of cloned genes, J. Mol. Biol, 189, 113-130.

9) Sutcliffe J. G., 1978, Nucleotide sequence of the ampicillin resistance gene of Escherichia coli plasmid pBR322, Proc. Natl. Acad. Sci. USA, 75, 3737-3741.

10) Zubay G., 1973, In vitro synthesis of protein in microbial systems, Ann. Rev. Geneti., 7, 267-287.

1. A method of discovering nucleic acids, in a biological, genomic or cDNA sample, that are associated with a pre-selected desired function, comprising:
preparing nucleic acids from the sample, wherein at least one of the prepared nucleic acids is unknown to the experimenter performing the method;
separating the prepared nucleic acids;
treating the separated nucleic acids in vitro to obtain transcripts;
optionally treating the transcripts in vitro to obtain proteins;
testing the transcripts or proteins for association with the desired function; and
identifying the nucleic acid that encodes the transcript or protein associated with the desired function.

2. A method of discovering nucleic acids, in a biological or genomic sample, that encode a desired function, comprising:
selecting a specific desired function;
preparing nucleic acids from the sample, wherein the existence, presence, identity, properties, or function of at least one of the prepared nucleic acids is unknown to the experimenter performing the method;
separating the prepared nucleic acids;
treating the separated nucleic acids in vitro to obtain transcripts;
treating the transcripts in vitro to obtain proteins;
testing the proteins for the desired function; and
identifying the nucleic acid that encodes the protein exhibiting the desired function.

3. A method of discovering nucleic acids, in a biological or genomic sample, that encode a desired RNA function, comprising:
selecting a specific desired RNA function;
preparing nucleic acids from the sample, wherein the existence, presence, identity, properties or function of at least one of the prepared nucleic acids is unknown to the experimenter performing the method;
separating the prepared nucleic acids;
treating the separated nucleic acids in vitro to obtain transcripts;
testing the transcripts for the desired function; and
identifying the nucleic acid that encodes the transcript exhibiting the desired function.

4. The method of claim 1, 2 or 3, wherein the existence, presence, identity, properties or function of more than one of the prepared nucleic acids is unknown to the experimenter performing the method.

5. The method of claim 4, wherein the identity, properties or function of more than one of the prepared nucleic acids is unknown to the experimenter performing the method.

6. The method of claim 5, wherein the function of more than one of the prepared nucleic acids is unknown to the experimenter performing the method.

7. The method of claim 4, wherein the function of more than one of the prepared nucleic acids is unknown to science.

8. The method of claim 7, wherein the identity, properties and function of more than one of the prepared nucleic acids are unknown to science.

9. The method of claim 8, wherein the existence, presence, identity, properties and function of more than one of the prepared nucleic acids are unknown to science.

10. The method of claim 4, wherein the existence, presence, identity, properties or function of at least the majority of the prepared nucleic acids is unknown to the experimenter performing the method.

11. The method of claim 10, wherein the identity, properties or function of at least the majority of the prepared nucleic acids is unknown to the experimenter performing the method.

12. The method of claim 11, wherein the function of at least the majority of the prepared nucleic acids is unknown to the experimenter performing the method.

13. The method of claim 4, wherein the function of at least the majority of the prepared nucleic acids is unknown to science.

14. The method of claim 13, wherein the identity, properties and function of at least the majority of the prepared nucleic acids are unknown to science.

15. The method of claim 14, wherein the existence, presence, identity, properties and function of at least the majority of the prepared nucleic acids are unknown to science.

16. The method of claim 4, wherein the sample is a genome-wide sample.

17. The method of claim 4, wherein the sample is a biological sample that includes or is extracted from at least one species or strain of organism.

18. The method of claim 17, wherein the organism is non-cultivable.

19. The method of claim 17, wherein the species or strain of organism is unknown to the experimenter performing the method.

20. The method of claim 17, wherein the sample is a biological sample that includes or is extracted from more than one species or strain of organism.

21. The method of claim 20, wherein the existence, presence, properties or identity of the more than one species or strain of organism is unknown to the experimenter performing the method.

22. The method of claim 20, wherein the properties and identity of the more than one species or strain of organism are unknown to the experimenter performing the method.

23. The method of claim 4, wherein the biological sample includes or is extracted from at least one organism whose identity is unknown to science.

24. The method of claim 23, wherein the biological sample includes or is extracted from more than one organism whose existence, presence, properties and identity are unknown to science.

25. The method of claim 4, wherein at least a majority of the prepared nucleic acids derive from at least one species or strain of organism whose presence or identity is unknown to the experimenter performing the method.

26. The method of claim 25, wherein at least a majority of the prepared nucleic acids derive from more than one species or strain of organism whose presence or identity is unknown to the experimenter performing the method.

27. The method of claim 20, wherein the method is performed without isolating the organisms from each other.

28. The method of claim 4, wherein the sample includes or derives from one or more extremophiles.

29. The method of claim 28, wherein the extremophiles are acidophiles that can grow in a pH lower than 2.

30. The method of claim 28, wherein the extremophiles are alkalinophiles that can grow in a pH higher than 11.

31. The method of claim 28, wherein the extremophiles are psychrophiles that can grow in temperatures below 0° C. to 4° C.

32. The method of claim 28, wherein the extremophiles are thermophiles that can grow in temperatures above 60° C. to 70° C.

33. The method of claim 28, wherein the extremophiles are barophiles that can grow at the pressures at the bottom of the ocean.

34. The method of claim 28, wherein the extremophiles are halophiles that can grow in a salt concentration of approximately 25-32 percent.

35. The method of claim 28, wherein the extremophiles are radiophiles that can grow in areas saturated with nuclear waste.

36. The method of claim 28, wherein the extremophiles are oligotrophs.

37. The method of claim 28, wherein the extremophiles are anaerobes.

38. The method of claim 28, wherein the extremophiles fall into more than one category of extremophile.

39. The method of claim 40, wherein the extremophiles are thermophilic barophiles that can grow in undersea thermal vents.

40. The method of claim 4, wherein separating the prepared nucleic acids initially comprises inserting the prepared nucleic acids into plasmidic vector molecules to form recombinant vectors, wherein the plasmidic vector molecules include a cloning site and an RNA polymerase promoter on at least one side of the cloning site.

41. The method of claim 40, wherein separating the prepared nucleic acids further comprises separating the recombinant vectors with a microorganism in which said promoter does not function.

42. The method of claim 1 or 2, wherein the step of treating the separated nucleic acids in vitro to obtain proteins is performed with a translation extract derived from an organism or organisms from the same family as the organism or organisms from which the sample derives.

43. The method of claim 42, wherein the translation extract is derived from an organism or organisms from the same genera as the organism or organisms from which the sample derives.

44. The method of claim 43, wherein the translation extract is derived from an organism or organisms from the same species as the organism or organisms from which the sample derives.

45. The method of claim 42, wherein the translation extract is prepared from eukaryotic cells.

46. The method of claim 42, wherein one or more of the prepared nucleic acids includes an amber codon and the translation extract includes a tRNA suppressor specific for that codon.

47. The method of claim 1 or 2, wherein the step of treating the separated nucleic acids in vitro to obtain proteins is performed with a universal translation extract derived from an organism or organisms from a different family, genera or species than the organism or organisms from which the sample derives.

48. The method of claim 1 or 2, wherein the step of treating the separated nucleic acids in vitro to obtain transcripts and the step of treating the transcripts in vitro to obtain proteins are coupled and occur simultaneously in the same reaction mixture.

49. The method of claim 1 or 2, wherein the step of treating the separated nucleic acids in vitro to obtain transcripts and the step of treating the transcripts in vitro to obtain proteins are temporally or physically distinct.

50. The method of claim 4, wherein the desired function is an enzymatic activity.

51. The method of claim 50, wherein the enzymatic activity is a member selected from the group consisting of oxidoreductase activity, transferase activity, hydrolase activity, lyase activity, isomerase activity and ligase activity.

52. The method of claim 3, wherein the desired RNA function is a member selected from the group consisting of a ribozyme function, a tRNA function, a Tm RNA function and a SI RNA function.

53. The method of claim 52, wherein the desired RNA function is a ribozyme function with an endonuclease activity.

54. The method of claim 1, wherein the testing step identifies a protein associated with the desired function and wherein the protein associated with the desired function is physically free from the nucleic acid that encodes it during and after the step of testing the protein for association with the desired function.

55. The method of claim 2, wherein the protein exhibiting the desired function is physically free from the nucleic acid that encodes it during and after the step of testing the protein for the desired function.

56. The method of claim 1 or 2, wherein the desired function is a function other than a binding affinity.

57. An apparatus for carrying out the method of claim 1, 2 or 3.

58. A kit for carrying out the method of claim 1, 2 or 3.

59. A kit for discovering nucleic acids, in a biological, genomic or cDNA sample, associated with desired functions, comprising:
one or more containers;
reagents for preparing nucleic acids from the sample, wherein at least one of the prepared nucleic acids is unknown to the experimenter using the kit;
vectors in which the prepared nucleic acids can be inserted;
reagents for inserting the prepared nucleic acids into the vectors to form recombinant vectors;
reagents for separating the recombinant vectors;
reagents for transcribing the separated recombinant vectors in vitro to obtain transcripts;
optionally, reagents for translating the transcripts in vitro to obtain proteins; and
reagents for testing the transcripts or proteins for a desired function.

60. A kit for discovering nucleic acids, in a biological or genomic sample, that encode desired functions, comprising:
one or more containers;
reagents for preparing nucleic acids from the sample, wherein the existence, presence, identity, properties or function of at least one of the prepared nucleic acids is unknown to the experimenter using the kit;
vectors in which the prepared nucleic acids can be inserted;
reagents for inserting the prepared nucleic acids into the vectors to form recombinant vectors;
reagents for separating the recombinant vectors;
reagents for transcribing the separated recombinant vectors in vitro to obtain transcripts;
reagents for translating the transcripts in vitro to obtain proteins; and
reagents for testing the proteins for a desired function.

61. A kit for discovering nucleic acids, in a biological or genomic sample, that encode desired RNA functions, comprising:
one or more containers;
reagents for preparing nucleic acids from the sample, wherein the existence, presence, identity, properties or function of at least one of the prepared nucleic acids is unknown to the experimenter using the kit;
vectors in which the prepared nucleic acids can be inserted;
reagents for inserting the prepared nucleic acids into the vectors to form recombinant vectors;
reagents for separating the recombinant vectors;
reagents for transcribing the separated recombinant vectors in vitro to obtain transcripts; and
reagents for testing the transcripts for a desired RNA function.

62. A kit for discovering nucleic acids, in a biological or genomic sample, that encode desired functions, comprising:
one or more containers;
nucleic acids prepared from the sample, wherein the existence, presence, identity, properties or function of more than one of the prepared nucleic acids is unknown to the experimenter using the kit;
recombinant vectors in which the unknown nucleic acids have been inserted;
transcripts of the recombinant vectors; and
one of either (i) reagents for translating the transcripts, (ii) proteins translated from the transcripts, (iii) reagents for testing the proteins for a desired function, (iv) or (v) a combination of any of (i)-(iv).

63. A kit for discovering nucleic acids, in a biological or genomic sample, that encode desired RNA functions, comprising:
one or more containers;
nucleic acids prepared from the biological or genomic sample, wherein the existence, presence, identity, properties or function of ore than one of the prepared nucleic acids is unknown to the experimenter using the kit;
recombinant vectors in which the unknown nucleic acids have been inserted;
transcripts of the recombinant vectors; and
reagents for testing the transcripts for a desired RNA function.
